(19) United States
(12) Patent Application Publication
(10) Pub. No.: US 2008/0183108 A1
(43) Pub. Date: Jul. 31, 2008

(54) REGISTRATION AND STABILITY TEST OF A KNEE BY RECORDING TWO POINTS ON THE KNEE

(76) Inventors: Herm Hansjoerg Huber, Munchen (DE); Blaine Warkentine, Droper, UT (US)

Correspondence Address: RENNER, OTTO, BOISSELLE & SKLAR, LLP, Nineteenth Floor, 1621 Euclid Avenue, Cleveland, OH, 44115-2191, US

(21) Appl. No.: 12/016,610

(22) Filed: Jan. 18, 2008

Related U.S. Application Data
(60) Provisional application No. 60/887,975, filed on Feb. 2, 2007.

(30) Foreign Application Priority Data
Jan. 19, 2007 (EP) EP 07 001 209.1

(51) Int. Cl. A61B 5/11

(52) U.S. Cl. 600/595; 600/587

(57) ABSTRACT

Disclosed is a method and device for testing the stability or mobility of the knee or knee joint and for calculating the isometry of the knee or knee joint. The method is performed by identifying and recording two points in the region of the knee or knee joint using a pointer, wherein the position and/or orientation of the points can be determined using a medical navigation system. By shifting the two recorded points by predetermined offset distances, a computer determines the axes of the tibia and the femur. By tracking the positions and orientations of tracking devices secured to the tibia and femur, the device can determine the stability or mobility of the knee joint.

### RELATED APPLICATION DATA

This application claims priority from Provisional Ser. No. 60/887,975, filed Feb. 2, 2007, and EP 07 001 209.1, filed Jan. 19, 2007, which are incorporated by reference.

### FIELD OF THE INVENTION

The present invention relates to a method and device for registering a knee or knee joint for image-guided surgery and for testing the stability or mobility of the knee or knee joint.

### BACKGROUND OF THE INVENTION

In order for a surgeon to have a better view of an operation on a knee, knee joint, or a knee ligament the operation is often performed while restricting the blood flow to the area. To avoid long-term damage to the surrounding tissue due to oxygen deprivation, the period of restricted blood flow should not be maintained for longer than 60 to 90 minutes. If a large number of points on the knee are to be recorded during the blood flow restriction period (e.g. for registering the knee for image-guided navigation or to obtain mobility information on the knee) the time available for the actual operation is greatly reduced. Accordingly, the method and device in accordance with the invention is intended to provide simple and quick knee or knee joint registration and to allow simple and quick determination of knee or knee joint mobility and stability.

### SUMMARY OF THE INVENTION

As noted above, the method and device in accordance with the invention enables registration and determination of the stability and/or mobility of a knee, a knee joint, or a knee ligament of a patient by identifying and recording just two reference points. The method can include a number of steps related to patient registration. One step entails identifying and recording the position of a first reference point on a tibia, such as a point on the anterior tibia or on the anteriorly lying portion of the tibia, using a pointing device. Both contact and/or non-contact pointing devices can be used, such as a mechanical pointer or a laser or infrared pointer. Another step entails using the pointer to identify and record a position of a second reference point on the tibia or patella, such as a point on an inferior end of the patella or knee-cap of the patient. Prior to the identifying and recording steps, a first tracking device can be attached or secured to the tibia of the patient or can be otherwise secured to the patient and a second tracking device can be attached or secured to the femur of the patient or can be otherwise secured to the patient. So that the pointer may be tracked by a navigation system, the method may include attaching or securing a tracking device, such as a reference star or the like, to the pointer. The tracking device may include markers that reflect or emit infrared radiation, but magnetically tracked devices can also be used. Using the tracking device, the medical navigation system can track the pointer by detecting the position and/or orientation of the tracking device. The positions and/or orientations of the tracking device can be used to determine the spatial position and/or orientation of the pointer. When the positions of the two reference points on the patient are identified and recorded, the navigation system can simultaneously track the tracking devices on the pointer and the patient. The navigation system, using the tracked devices, can determine the position and/or orientation of the pointer and a pointer tip, along with the positions and/or orientation of the patient tracking devices. Thus, the positions and orientations of the tibia, femur, and pointer may be known to the navigation system. From the positions and/or orientations of the pointer and patient tracking devices, a computer (connected to or integrated in the navigation system) can determine the positions of the first and second reference points with respect to a first coordinate system defined in a relationship with the tibia and/or with respect to a second coordinate system defined in a relationship with the femur. The first coordinate system, for example, can lie in the center of the knee, in the region of the center of the knee, or in the region of the first tracking device. The second coordinate system can lie in the center of the knee, in the region of the center of the knee, or in the region of the second tracking device.

Once the positions of the first and second reference points are known to the navigation system, with the assistance of the computer the position of the first reference point can be shifted or offset by a first predetermined amount into a position of a first axial point and the position of the second reference point can be shifted or offset by a second predetermined amount into a position of a second axial point. The first reference point is preferably shifted or offset into the first axial point by an amount in the range of 1 to 3 cm, for example 1.5 cm, and the second reference point is preferably shifted or offset by an amount in the range of 2 to 5 cm, for example 3 cm. The shift or offset amounts may be determined based on the age, size, weight, or sex of the patient or other relevant patient specific criteria. The first reference point can be shifted into the first axial point (that preferably lies within the tibia) in a direction that can be parallel to or collinear with the direction and/or orientation of the pointer. The direction and/or orientation of the pointer may be determined by the navigation system while identifying and recording the position of the first reference point. In other words, the navigation system can determine the position of the first axial point by recording the position of the first reference point with the pointer and then use the position and/or orientation of the pointer at the instant of recording, to shift the first reference point a predetermined distance in the known direction and/or orientation of the pointer. The position of the second reference point may be shifted into a position of the second axial point (that preferably lies within the knee joint) in the same manner. The position of the first reference point may be shifted by, for example about 1.5 cm, and the position of the second reference point may be shifted by, for example by about 3 cm, into the respective positions of the first and second axial points. From these known axial point positions, it is possible to determine or approximate the mechanical tibia axis, by connecting the positions of the first and second axial points.

Once the registration is complete and the positions of the reference points and the axial points have been determined with respect to the tracking devices, the tibia and the femur may be placed in a first relative position, e.g. an extended position. In this first relative position, the femur approximates a linear extension of the tibia such that the relative angle between the tibia and femur approaches an angle of 180°. In this position, the femur is preferably oriented neutrally relative to the tibia by compensating for an existing rotation between the tibia and the femur. In other words, such that the tibia lies untwisted relative to the femur. While in this extended and untwisted position, the navigation system may determine the positions of the two tracking devices and therefore the positions of the reference points and axial points. With all of the positions known to the navigation system, the system may calculate the relative distance or length between the reference points and between the axial points.

Additionally, while still in the extended position in which the tibia and the femur are oriented rotationally neutral relative to each other, a mobility test can be performed to determine the mobility of the tibia relative to the femur. During the mobility test, the navigation system may determine the positions of the first and second axial points relative to each other by determining the positions and/or orientations of the first and second tracking devices. Moreover, during the mobility test three aspects of mobility may be tested via: a rotational mobility test, a translational mobility test, and a flexion mobility test. All three tests assist in determining the mobility of the tibia and the femur relative to each other. In the rotational mobility test, the tibia may be rotated about a longitudinal axis relative to the femur, and the rotation angle by which the tibia can be rotated to its maximum rotation relative to the femur may be determined by determining the position of the first axial point relative to the position of the second axial point. In the translational mobility test, the tibia may be shifted or moved anteriorly, perpendicular to the longitudinal axis relative to the femur, and the translation amount by which the tibia is shifted to a maximum translation relative to the femur may be determined by determining the position of the first axial point relative to the position of the second axial point. In the flexion mobility test, the tibia may be bent or flexed relative to the femur, and the flexion angle by which the tibia can be bent or flexed to a maximum bent position relative to the femur may be determined by determining the position of the first axial point relative to the position of the second axial point.

After the mobility test or tests are performed at a full extension of the tibia and the femur relative to each other, the tibia and the femur are bent into a second relative position by a predetermined second angular value. (At full extension, a first angular value is 0°.) The second angular value, can be for example 15° to 45° (more particularly 30°), relative to the extended position. In other words, the tibia and femur are placed in a second relative position such that the relative angle between the bones may be about 150°. In this second relative position, the mobility test is performed again, and during this second mobility test, the navigation system may again determine the positions of the first and second axial points relative to each other. Again, the navigation system may determine the positions and/or orientations of the first and second tracking devices to determine the positions of the first and second axial points. Before the second mobility test is performed, the tibia and the femur are preferably placed in a neutral rotational position relative to each other. The second mobility test may also include individual rotational, translational, and flexion mobility tests.

After the second mobility test, the tibia and the femur may be bent by a predetermined third angular value relative to the extended position, into a third relative position to one another. The third angular value, can be an additional 60°, such that when added to the second angular value of 30°, the tiba and the femur are 90° from a fully extended position. (The third relative angular position of the tibia and the femur is also 90°.) Preferably, the third relative position between the tiba and femur may be in a range from 75° to 105°. In this third relative position, the mobility test may be performed again, and the navigation system may again determine the positions of the first and second axial points by determining the positions and/or orientations of the first and second tracking devices. Before this third mobility test, the tibia and the femur are preferably placed in a neutral rotational position relative to each other and the test may be performed in a similar manner to the other tests.

The mobility tests performed in the different positions may proceed in the same manner or can differ from each other. For example, all three mobility tests: the rotational, translational, and flexion may be performed in each of the three relative positions, or individual tests can be selected and performed.

As noted above, the stability and mobility of the knee, knee joint, or knee ligament may be ascertained from the determined positions of the first and second axial points, and their relative distances from one another during the tests.

In other words, the method in accordance with the invention can include placing the tibia and the femur in a neutral position relative to each other before the mobility tests, preferably in a neutral rotational and translational position. Determining the positions of the axial points relative to each other in the neutral position by determining the position and/or orientation of the tracking devices using the navigation system. Determining the maximum mobility of the knee or knee joint may be determined during the mobility tests, in the respective maximum positions. If the respective maximum deflection has been reached, such as the maximum rotation, translation, or flexion of the tibia relative to the femur, the position of the axial points relative to each other at the maximum deflection may be determined by the navigation system by determining the position and/or orientation of the tracking devices.

The positions of the axial points are preferably determined in the neutral start position and in the maximum possible end position during the mobility test at the full extension of the tibia and the femur relative to each other. The positions of the axial points in the neutral start position and in the maximum possible end position are again preferably determined in the position bent from full extension by the second angular value, for example 30°, and by the third angular value 60°, for a total relative position of 90°. The stability or mobility of the knee or knee joint is preferably determined from the positions of the axial points in these relative positions.

The navigation system can also determine the position and/or orientation of the tibia axis that may be determined or approximated from the positions of the first and second axial points. By determining the positions and/or orientations of the first and second tracking devices, the positions and orientations of the tibia axis may be determined in the start and end positions during the mobility tests (that may be performed at relative angular positions of 180°, 150°, and 90°). The mobility of the knee or knee joint may be ascertained from the positions and/or orientations of the approximated tibia axis and femur axis. The femur axis may be approximated from the tibia axis by extrapolating or reflecting the tibia axis, at the second axial point (the center point of the knee), when the knee is fully extended. Once defined, the femur axis may be tracked by the navigation system using the tracking device attached thereto.

The tibia axis is preferably used as an axis of the first coordinate system with respect to which the reference points and axial points can be defined or registered. An axis which runs parallel to the orientation of the pointer while recording the first reference point is preferably used as another axis of the first coordinate system. The femur axis is preferably used as an axis of the second coordinate system with respect to which the reference points and axial points can be defined or registered. An axis which runs parallel to the orientation of the pointer while recording the second reference point is preferably used as another axis of the second coordinate system.

In accordance with the invention, there is also provided a computer program that, when it is loaded onto a computer or is running on a computer, performs the method described above. There is also provided a program storage medium that stores the computer program, and/or a computer software product that includes the computer program.

A device in accordance with the invention for determining the stability and/or mobility of a knee or knee joint of a patient can include a medical navigation system having infrared cameras that can emit and detect infrared radiation, or cameras that can simply detect emitted infrared radiation. Alternatively, a magnetic-tracking based medical navigation system may be used. A computational unit, such as a computer or a processor, can be connected wirelessly or wired to the navigation system or can be integrated into the navigation system such that information can be exchanged between the navigation system and the computational unit. The navigation system can detect the position and/or orientation of the pointer during the identification and recording process by using its infrared cameras to detect the infrared radiation reflected or emitted by the tracking device arranged on the pointer. With the position and/or orientation of the pointer tracked and known, the navigation system can identify and record the positions of the first reference point on a tibia of the patient and the second reference point on the tibia or patella of the patient when the pointer is located at those respective points.

From the detected position and/or orientation of the pointer, the computational unit can determine the positions of the first and second reference points with respect to a first coordinate system defined in relation to the tibia and/or with respect to a second coordinate system defined in relation to the femur. If the positions of the two reference points have been determined and are known to the computational unit, the computational unit can shift or offset the position of the first reference point by a first predetermined amount between 1 and 3 cm, for example 1.5 cm, into a first axial point, and can shift the position of the second reference point by a second predetermined amount between 2 to 5 cm, for example 3 cm, into a second axial point.

To use the device to perform a mobility test, the tibia and the femur can be placed in a position in which they are situated in complete extension relative to each other, (i.e. placing the tibia and the femur at an angle of approximately 180° relative to each other). In this position, a mobility test can be performed in order to determine the mobility of the tibia relative to the femur. When the mobility test is performed, the computational unit may determine the positions of the first and second axial points relative to each other by determining the positions and/or orientations of a first tracking device attached or secured to the tibia and a second tracking device attached or secured to the femur. The positions of the axial points may be determined at the beginning of the mobility test or at the beginning of each individual test of the mobility test and at the respective maximum deflections. The determined positions of the axial points during the mobility test may be calculated in the computational unit at each positioning of the patient's tibia and femur.

The tibia and the femur may then be bent by a second angular value, for example by 30°, relative to the extended position. In this position, a second mobility test may be performed. When the second mobility test is performed, the computational unit can determine and calculate the positions of the first and second axial points relative to each other by determining the positions and/or orientations of the first and second tracking devices. The computational unit may determine and calculate the start and end positions of the axial points during the second mobility test.

The tibia and the femur may then be bent to a third position relative to each other in a range from 75° to 105°, preferably about 90°. In this position, a third mobility test may be performed. When the third mobility test is performed, the computational unit can determine and calculate the positions of the first and second axial points by determining the positions and/or orientations of the first and second tracking devices. The computational unit may determine and calculate the start and end positions of the axial points during the third mobility test.

The device in accordance with the invention may include a display device, such as a screen, monitor, or display, that is connected to the navigation system and/or the computational unit. On the display device, representations of the tibia, the femur, the reference points, the axial points, and any other determined data can be displayed.

### BRIEF DESCRIPTION OF THE DRAWINGS

The forgoing and other features of the invention are hereinafter discussed with reference to the drawings.

FIG. 1a illustrates a recording of a first reference point on a tibia of a patient using a pointer.

FIG. 1b illustrates a recording of the first reference point on a tibia of the patient, in a position with the tibia positioned at 180° relative to a femur.

FIG. 2a is a top view of a recording of a second reference point on a patella of the patient using a pointer in the position of the tibia positioned at 180° relative to the femur.

FIG. 2b is a lateral view of a recording of a second reference point on a patella of the patient using a pointer in the position of the tibia positioned at 180° relative to the femur.

FIG. 3a shows the start position of a mobility test in a position in which the tibia is positioned at 30° from a completely extended position relative to the femur, at 150° relative to the femur.

FIG. 3b shows the performance of a mobility test with the tibia in the position shown in FIG. 3a, relative to the femur.

FIG. 4a shows the start position of a mobility test in a position in which the tibia is bent by 90° from a completely extended position relative to the femur.

FIG. 4b shows the performance of a mobility test with the tibia in the position shown in FIG. 4a, relative to the femur.

### DETAILED DESCRIPTION

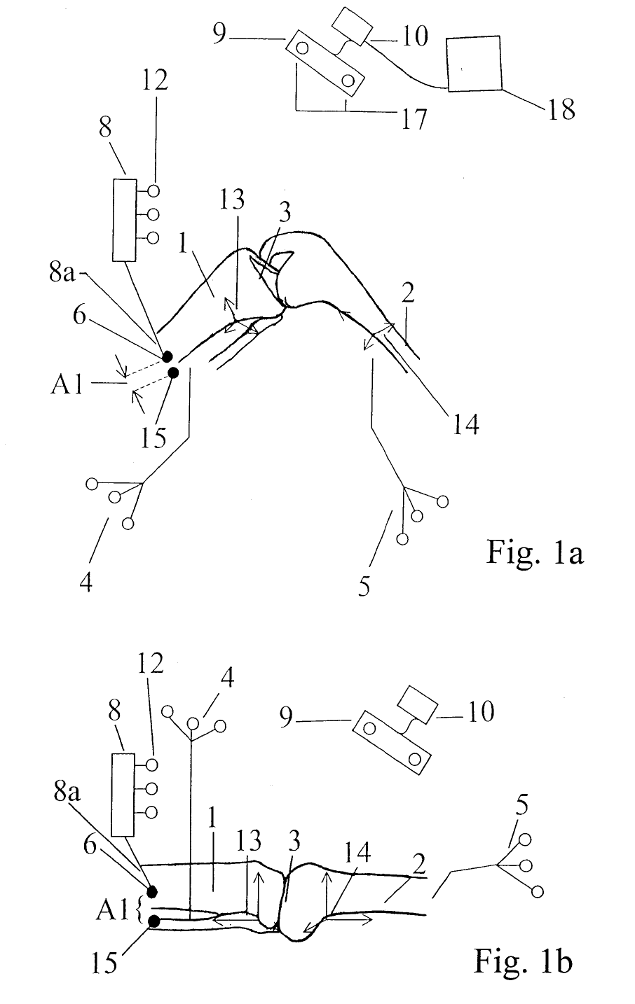

FIGS. 1a and 1b show a femur 2 and an anterior region of a tibia 1 of a patient, wherein two reference stars 4, 5 are secured or attached to each of both the tibia 1 and the femur 2 respectively. A point on the anterior tibia 1 or a point in an anterior region of the tibia 1 may be identified and recorded as a reference point 6, using a pointer 8. A reference star 12 may be also attached or secured to the pointer 8, such that the position and/or orientation of the reference star 12 can be detected, tracked, and determined using a navigation system 9. Using the determined position and/or orientation of the reference star 12, the navigation system 9 can use a pair of cameras 17 to determine the position and/or orientation of the pointer 8, such that the navigation system 9 can also determine the position and/or orientation of the tip 8a of the pointer 8. The navigation system 9 can determine the position and/or orientation of the tip 8a of the pointer 8 during the identification and recording process of the reference point 6 on the tibia 1 such that the navigation system 9 can determine the position of the reference point 6 on the anterior tibia 1. Thus, the navigation system 9 has in memory or knows both the position of the reference point 6 on the anterior tibia 1 and the position and/or orientation of the pointer 8, in particular the tip 8a of the pointer 8, during the recording process of the reference point 6. Additionally, from this information the navigation system 9 and/or a computational unit 10 connected to the navigation system 9, can determine a position of an axial point 15 of the tibia 1 by virtually shifting the position of the reference point 6 posteriorly by a predetermined first amount A1, such as an amount between 0.5 and 2.5 cm. The position of the reference point 6 can be shifted by the amount A1, for example 1.5 cm, in a direction parallel to or collinear with the orientation of the pointer 8 during the identification and recording process of the reference point 6.

Also shown in FIGS. 1a and 1b are a first coordinate system 13 that may be defined with an axis that runs parallel to the tibia 1 and an axis that runs parallel to the orientation of the pointer 8 while recording the first reference point 16. Also shown is a second coordinate system 14 that may be defined with an axis that runs parallel to the femur 2 and an axis that runs parallel to the orientation of the pointer 8 while recording a second reference point 7.

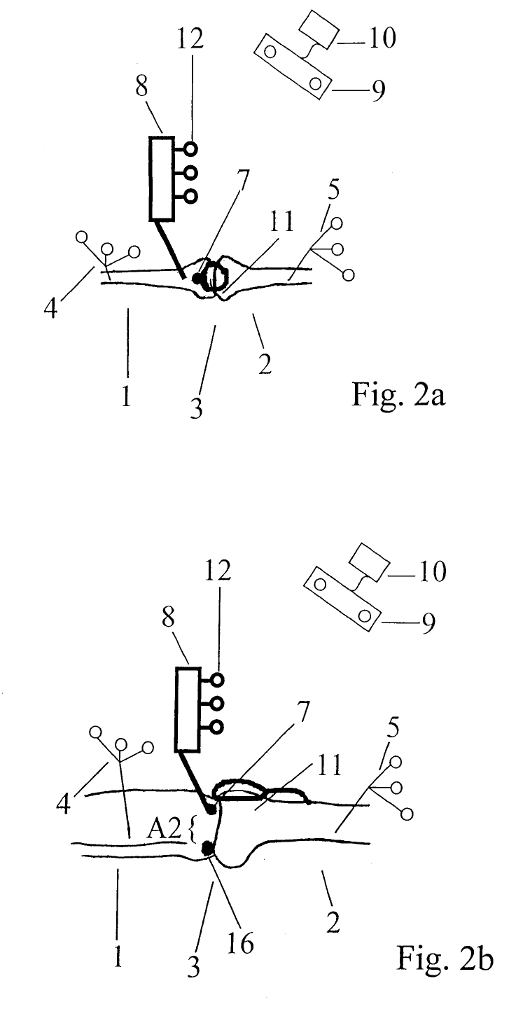

FIGS. 2a and 2b show how a reference point 7 on an inferior end of a patella 11 (inferior patella pole) is identified and recorded using the tip 8a of the pointer 8. The position of the reference point 7 on the patella 11 can be determined by the navigation system 9, and/or by the computational unit 10 connected to the navigation system 9 (e.g., by determining the position and/or orientation of the pointer 8 or its tip 8a during the recording process of the reference point 7 on the patella 11). To determine this position, the navigation system 9 can use the determined position and/or orientation of the reference star 12 attached or secured to the pointer 8. During the recording process of the reference point 7 on the patella 11, the tibia 1 is preferably positioned in complete extension relative to the femur 2, such that the tibia 1 and the femur 2 lie at an angle approximating 180° relative to each other. The navigation system 9 can determine the position of another axial point 16 near the center of the knee 3 from the position of the reference point 7 situated on the patella 11, by virtually shifting the reference point 7 by a predetermined second amount A2, such as a value between 2 and 4 cm. The reference point 7 can be posteriorly shifted by the second predetermined amount A2 in a direction which is parallel to or collinear with the orientation of the pointer 8, while recording the reference point 7 on the patella 11. The second axial point 16 may be located at a position in the interior of the knee 3 near the knee's center.

Both the first and second amounts A1 and A2 can be selected based on patient parameters such as the size of the patient or the bone thickness of the patient. In the case of a child, for example, a value of 0.5 cm may be used as the first amount A1 and a value of 1.5 or 2 cm may be used as the second amount A2, while in the case of a large, athletic person, a value of 2 cm or more may be used as the first amount A1 and a value of 4 cm or more may be used as the second amount A2. When the axial point 15 of the tibia 1 and the axial point 16 (near the center of the knee 3) are virtually connected to each other in the computational unit 10, an approximation of the tibia's mechanical axis may be determined. The computational unit 10 can also determine an approximation of the femur axis from the tibia axis, by reflecting or extrapolating the tibia axis at the center of the knee, when the tibia 1 and the femur 2 are arranged in complete extension.

In the position in which the tibia 1 and the femur 2 are arranged in complete extension relative to each other (or are positioned at 180° relative to each other), a first mobility test may be performed. The mobility test may include a rotational mobility test, a translational mobility test, and a flexion mobility test, in each of which the tibia 1 is maximally deflected relative to the femur 2. Before the beginning of the mobility test, the tibia 1 may be placed in a neutral rotational position relative to the femur 2, such that the tibia 1 is not twisted relative to the femur 2. At the beginning of the mobility test, the position of the axial points 15 and 16 and/or the positions of the tibia axis and/or of the femur axis may be determined.

In each of the mobility tests, the tibia 1 may be maximally deflected relative to the femur 2, and the position of the axial points 15 and 16 and/or of the tibia axis and/or of the femur axis may be determined at the point of maximum deflection. In the rotational mobility test, the tibia 1 may be rotated relative to the femur 2 until a maximum rotational deflection is reached, and the position of the axial points 15, 16 and/or the positions of the tibia axis and/or of the femur axis may be determined at the point of maximum rotational deflection. In the translational mobility test, the tibia 1 may be shifted relative to the femur 2 until a maximum translational deflection is reached, and the position of the axial points 15 and 16 and/or the positions of the tibia axis and/or of the femur axis may be determined at the point of maximum translational deflection. In the flexion mobility test, the tibia 1 may be extended or flexed relative to the femur 2 until a maximum flexion deflection is reached, and the position of the axial points 15 and 16 and/or the positions of the tibia axis and/or of the femur axis may be determined at the point of maximum flexion deflection. The first mobility test therefore provides a number of calculated positions of the axial points 15 and 16 and/or the positions of the tibia axis and/or of the femur axis.

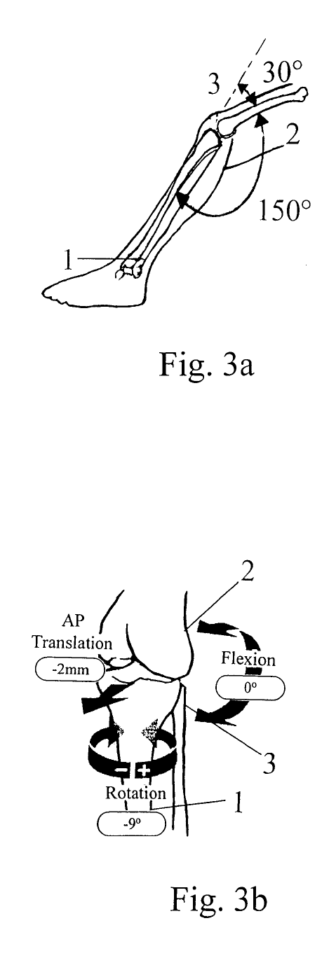

After the first mobility test has been performed, the tibia 1 may be positioned, as shown in FIG. 3a, out of the extended start position relative to the femur 2 by a predetermined second amount, for example 30°. In this position, a second mobility test—which can proceed similarly to the first mobility test—may be performed, as shown in FIG. 3b. Also, during the second mobility test, the positions of the axial points 15 and 16 and/or the positions of the tibia axis and/or of the femur axis are preferably determined in a start position of the mobility test and in a position of maximum deflection. This results in the calculation of a number of positions of the axial points 15 and 16 and/or the positions of the tibia axis and/or of the femur axis.

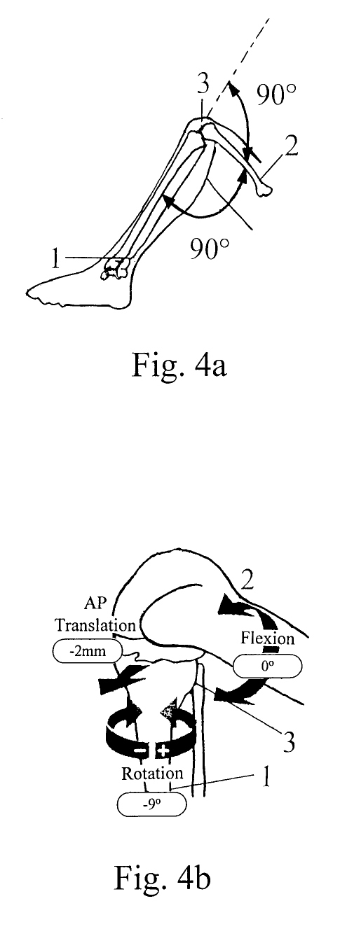

After the second mobility test has been performed, the tibia 1 may be bent relative to the femur 2, by a predetermined third amount, for example by another 60°, such that the tibia 1 and the femur 2 are positioned at an angle of 90° relative to each other, as shown in FIG. 4a. In this position, a third mobility test—which can proceed similarly to the first and/or second mobility test—may be performed, as shown in FIG. 4b. Also during the third mobility test, the positions of the axial points 15 and 16 and/or the positions of the tibia axis and/or of the femur axis are preferably determined in a start position of the mobility test and in a position of maximum deflection. This results in the calculation of a number of positions of the axial points 15 and 16 and/or positions of the tibia axis and/or of the femur axis.

The navigation system 9 can determine the stability and/or mobility of the knee 3, knee joint, or knee ligaments from a portion of the determined positions or from all the determined positions of the axial points 15 and 16 and/or the positions and/or orientations of the tibia axis and/or of the femur axis during the mobility tests. The determined stability or mobility of the knee joint can be determined before a knee joint operation and can be used to restore or maintain the previous or originally existing stability or mobility of the knee 3 during or after a knee joint operation. It may also be used to check how far the stability or mobility of the knee 3 or knee joint (after the operation) deviates from the stability or mobility before the operation.

A display device 18, such as a screen, monitor, or a display, is shown connected to the navigation system 9 and/or the computational unit 10. On this display device 18, representations of the tibia 1, the femur 2, the reference points 6, 7, the axial points 15, 16, and any other determined data can be displayed.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, software, computer programs, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A method for determining stability and mobility of a knee or knee joint of a patient said knee or knee joint including a tibia, femur, and patella comprising:
securing or attaching a first tracking device to the tibia of the patient and a second tracking device to the femur of the patient;
recording a position of a first reference point on the tibia using a pointing device and a position of a second reference point on the tibia or the patella using the pointing device, said pointing device including a third tracking device secured or attached thereto;
using a navigation system to detect and determine the position and/or orientation of the pointing device during the recording process of the positions of the first and second reference points, using the third tracking device;
offsetting the position of the first reference point by a first predetermined amount into a position of a first axial point, and offsetting the position of the second reference point by a second predetermined amount into a position of a second axial point;
placing the tibia and the femur in a plurality of positions relative to each other;
performing a mobility test on the knee joint in the plurality of different positions of the tibia relative to the femur, wherein the navigation system determines the positions of the first and second axial points relative to each other during the test by determining the positions and/or orientations of the first and second tracking devices; and
ascertaining the stability and mobility of the knee or knee joint from the determined positions of the first and second axial points.

2. The method according to claim 1, wherein a point on the anterior tibia is used as the first reference point.

3. The method according to claim 1, wherein a point on the inferior end of the patella is used as the second reference point.

4. The method according to claim 1, wherein the tibia axis is approximated by connecting the positions of the first and second axial points.

5. The method according to claim 1, wherein the positions of the first and second reference points are determined from the position and/or orientation of the pointing device with respect to a first coordinate system defined in a relationship with the tibia and/or with respect to a second coordinate system defined in a relationship with the femur.

6. The method according to claim 1, further including the steps of:
bending the tibia and the femur to a first angular position relative to one another;
performing the mobility test at the first angular position;
bending the tibia and the femur by a second angular position relative to one another;
performing the mobility test at the second angular position;
bending the tibia and the femur by a third angular position relative to one another; and
performing the mobility test at the third angular position.

7. The method according to claim 6, wherein the first angular position is approximately 180°, the second angular position is in a range of 135° to 165°, and the third angular position is in a range of 75° to 105°.

8. The method according to claim 4, wherein the femur axis is approximated from the tibia axis by extrapolating or reflecting the tibia axis at full extension.

9. The method according to claim 1, wherein the mobility test includes performing a rotational mobility test, a translational mobility test and a flexion mobility test of the tibia and the femur relative to each other.

10. The method according to claim 1, wherein the mobility test includes a rotational mobility test wherein the rotational angle by which the tibia can be rotated relative to the femur is determined using the relative positions of the first and second axial points.

11. The method according to claim 1, wherein the mobility test includes a translational mobility test wherein the translational amount by which the tibia can be shifted relative to the femur is determined using the relative positions of the first and second axial points.

12. The method according to claim 1, wherein the mobility test includes a flexion mobility test wherein the flexion angle by which the tibia can be flexed relative to the femur is determined using the relative positions of the first and second axial points.

13. The method according to claim 1, wherein the tibia and the femur are placed in a neutral rotational position relative to each other before the mobility test.

14. The method according to claim 5, wherein the second axial point, that approximates a center of the knee or knee joint, is used as the origin of the first and the second coordinate systems.

15. The method according to claim 5, wherein the tibia axis is used as an axis of the first coordinate system, and an axis which runs parallel to the orientation of the pointing device while recording the position of the first reference point is used as another axis of the first coordinate system.

16. The method according to claim 5, wherein the femur axis is used as an axis of the second coordinate system, and an axis which runs parallel to the orientation of the pointing device while recording the position of the second reference point is used as another axis of the second coordinate system.

17. The method according to claim 1, wherein the first predetermined amount is in a range of 1 to 3 cm and the second predetermined amount is in a range of 2 to 5 cm.

18. The method according to claim 1, wherein the first and second tracking devices are reference stars.

19. A computer-readable medium storing a computer program, wherein when the program is loaded onto a medical navigation system or a computational unit and executed assists a physician in performing the following method on a knee or knee joint of a patient, said knee or knee joint including a tibia, femur, and patella:
securing or attaching a first tracking device to the tibia of the patient and a second tracking device to the femur of the patient;
recording a position of a first reference point on the tibia using a pointing device and a position of a second reference point on the tibia or the patella using the pointing device, said pointing device including a third tracking device secured or attached thereto;
using a navigation system to detect and determine the position and/or orientation of the pointing device during the recording process of the positions of the first and second reference points, using the third tracking device;
offsetting the position of the first reference point by a first predetermined amount into a position of a first axial point, and offsetting the position of the second reference point by a second predetermined amount into a position of a second axial point;
placing the tibia and the femur in a plurality of positions relative to each other;
performing a mobility test on the knee joint in the plurality of different positions of the tibia relative to the femur, wherein the navigation system determines the positions of the first and second axial points relative to each other during the test by determining the positions and/or orientations of the first and second tracking devices; and
ascertaining the stability and mobility of the knee or knee joint from the determined positions of the first and second axial points.

20. A device for determining the stability and mobility of a knee or knee joint of a patient, said knee of knee joint including a tibia, femur, and patella, comprising:
a navigation system having at least one infrared camera;
a first tracking device and a second tracking device, adapted to be attached or secured to a patient;
a pointing device having a third tracking device; and
a computational unit that is connected to the navigation system or is integrated into the navigation system;
wherein the navigation system is adapted to detect the position and/or orientation of the pointing device using the third tracking device while the pointing device is used to identify and record a position of a first reference point on a tibia and a position of a second reference point on the tibia or the patella using the third tracking device;
wherein the computational unit is adapted to calculate an offset position of the first reference point by a first predetermined amount into a position of a first axial point and calculate an offset position of the second reference point by a second predetermined amount into a position of a second axial point;
wherein the computational unit is further adapted to determine the positions of the first and second axial points relative to each other during a mobility test by determining the positions and/or orientations of the first tracking device that is secured or attached to the tibia and the second tracking device that is secured or attached to the femur; and
wherein the computational unit is further adapted to ascertain the stability and mobility of the knee or knee joint from the determined positions of the first and second axial points.

21. The device according to claim 20, wherein the device also comprises a display device which is connected to the navigation system and/or the computational unit and on which the tibia, the femur, the reference points and the axial points can be displayed.

22. The device according to claim 20, wherein the computational unit is adapted to determine the positions of the first and second reference points with respect to a first coordinate system defined in a relationship with the tibia and/or with respect to a second coordinate system defined in a relationship with the femur.

23. The device according to claim 20, wherein the first and second tracking devices are reference stars.

24. The device according to claim 20, wherein the pointing device is a contact pointing device.

25. The device according to claim 20, wherein the pointing device is a non-contact pointing device.
